(19) United States
(12) Patent Application Publication
(10) Pub. No.: US 2017/0307627 A1
(43) Pub. Date: Oct. 26, 2017

(54) HIGH RESOLUTION IMAGING OF TISSUE PROTEINS

(71) Applicants: ARATOME, LLC, Menlo Park, CA (US); THE BOARD OF TRUSTEES OF THE LELAND STANFORD JUNIOR UNIVERSITY, Palo Alto, CA (US)

(72) Inventors: Gordon WANG, San Jose, CA (US); Jay TRAUTMAN, Los Altos, CA (US)

(21) Appl. No.: 15/481,141

(22) Filed: Apr. 6, 2017

Related U.S. Application Data
(63) Continuation of application No. PCT/US15/54771, filed on Oct. 8, 2015.
(60) Provisional application No. 62/061,622, filed on Oct. 8, 2014.

(51) Int. Cl. G01N 33/68; G01N 33/53; G01N 33/533; G01N 33/537

(52) U.S. Cl. CPC G01N 33/6803; G01N 33/5375; G01N 33/53; G01N 33/533

(57) ABSTRACT

The present invention provides labeling method for Array Tomography (AT) and immunohistochemistry (IHC) that enables automated analysis many tens-to-hundreds of proteins in a manner that minimizes tissue degradation, increases data fidelity, and substantially increases throughput and reduces cost. Also included are methods for automated acquisition of AT and IHC data.

### CROSS REFERENCE TO RELATED APPLICATIONS

This application claims the benefit under 35 U.S.C. 119(e) of U.S. Provisional Patent Application No. 62/061,622, filed Oct. 8, 2014, which is herein incorporated by reference in its entirety.

### BACKGROUND

Array tomography (AT) is an imaging method for quantitative, molecular analysis of protein expression in the context of the three-dimensional tissue architecture. Reconstruction of the detailed architecture of tissue is accomplished by sectioning thin tissue slices (˜21-1000 nm), immunofluorescence labeling, imaging at, or near, the diffraction limit, and assembling the three-dimensional data, in silica. Antibodies can be stripped, and stain-and-imaging cycles repeated many times, to build up three-dimensional, proteomic data sets.

### SUMMARY

An object of the present invention is to provide methods of performing array tomography on an intact tissue sample to facilitate spatially resolved identification of a plurality of proteins in the tissue.

Provided herein are methods comprising: contacting an intact tissue sample with at least one binding ligand that binds a particular protein, wherein said binding ligand is linked to a nucleic acid label; and detecting said nucleic acid label, thereby detecting the presence of said protein in the tissue sample. In an aspect, the methods may comprise contacting the intact tissue with a plurality of binding ligands, wherein each ligand that binds a specific protein is linked to a unique nucleic acid label. In some cases each binding ligand in the plurality of binding ligands may bind a different protein.

Provided herein are methods comprising: contacting an intact tissue sample with at least one binding ligand which is an antibody that binds a particular protein, wherein said antibody is linked to a nucleic acid label; and detecting said nucleic acid label, thereby detecting the presence of said protein in the tissue sample. In an aspect, the methods may comprise contacting the intact tissue with a plurality of antibodies, wherein each antibody that binds a specific protein is linked to a unique nucleic acid label. In some cases each antibody in the plurality of antibodies may bind a different protein. The antibodies may be of the same or different isotypes, and the nucleic acid labels may comprise DNA and/or RNA.

In some methods described herein, at least one binding ligand is a peptide or nucleic acid affinity reagent. Affinity reagents that are used in the methods described herein include for instance designed ankyrin repeat proteins (20 kD) and anticalins (20 kD) that have been evolved to hind particular proteins while maintaining stability. Additional affinity reagents that are used in the methods described herein include for instance cysteine knottin scaffold (20-50 amino acids), cyclic peptides (17 amino acids), fynomers (63 amino acids), affitin (65 amino acids), sso7d (63 amino acids) and fibronectins (94 amino acids) which are designed to bind particular proteins. In some cases, at least one binding ligand is an affibody that has been designed to bind a particular protein. The 45 amino acid stretch of T7 phage gene 2 protein (Gp2) or fragments thereof may also be used as a binding ligand in some methods described herein.

In some of the methods described herein, the intact tissue may be embedded in a resin such that the tissue can be sliced into sections of thickness between 20 and 1000 nm. In some cases, the method may not comprise dehydration or resin-embedding of the intact tissue.

In some of the methods described herein, detection comprises exposing the tissue to detection labels unique for each nucleic acid label. Each detection label may comprise at least one nucleic acid oligomer comprising a sequence which is complementary to a sequence of at least one nucleic acid label. In some cases, the detection label comprises at least one detection tag. The detection label may also comprise a plurality of detection tags. In some cases one or more of the detection tags maybe attached to the oligomer. A detection tag may be attached to an oligomer by a cleavable or a non-cleavable linker. In some cases a plurality of detection tags are attached to the oligomer between seven and thirty bases apart from each other. In some instances, the plurality of detection tags may comprise between two and ten detection tags.

In some of the methods described herein, each detection tag may comprise a fluorescent tag. In some cases the fluorescent tag may be a quantum dot (QD). In some cases the fluorescent tag may be at least one fluorescent dye. In some exemplary instances, the fluorescent dye may comprise at least one of coumarin rhodamine, xanthene, fluorescein and cyanine.

Some of the methods described herein may not comprise the detection of a secondary antibody.

In some cases, the methods described herein, may comprise a detection step that comprises determining the sequence of each nucleic acid label. In some cases the sequence of each nucleic acid label may be determined by sequencing by synthesis. In some instances, the sequence of each nucleic acid label may be determined by sequencing by hybridization. Sequencing by hybridization may involve use of the tag hybridization method described herein.

Some methods described herein comprise use of a microfluidic chamber. Some methods may be fully automated.

In some methods, the at least one binding ligand maybe cross-linked to the tissue.

Some methods described herein may be used to identify the protein composition of the tissue sample.

A method described herein may comprise contacting the intact tissue sample with a plurality of binding ligands, wherein each type of binding ligand that binds a specific protein is linked to a unique nucleic acid label. In some cases the binding ligands are antibodies, or peptide or nucleic acid affinity reagents designed to bind specific proteins as described above.

In the methods described herein, the detection of the nucleic acid label may be spatially resolved.

In some of the methods described herein, contacting the tissue with a binding ligand may comprise application of an electric field. An electric field may also be applied during the hybridization of oligomers in methods comprising such hybridization.

Provided herein is a system for identifying the protein composition of an intact tissue comprising: an intact tissue sample; at least one binding ligand that binds a particular protein, wherein said binding ligand is linked to a unique nucleic acid label; and a detector for detection of said nucleic acid label. In some systems, the intact tissue is resin embedded. In some systems, the intact tissue may not be dehydrated. A system described herein may comprise a plurality of binding ligands, wherein each binding ligand that binds a specific protein is linked to a unique nucleic acid label. In some systems, detection may comprise exposing the tissue to detection labels unique for each nucleic acid label. In certain systems, each detection label may comprise at least one nucleic acid oligomer comprising a sequence which is complementary to a sequence of at least one nucleic acid label. In some cases the binding ligands are antibodies, or peptide or nucleic acid affinity reagents designed to bind specific proteins as described above.

In some systems described herein, the detection label may comprise at least one detection tag. Some detection tags may comprise a fluorescent tag. In some systems, the detection may comprise determining the sequence of each nucleic acid label. The sequence of the nucleic acid label may be determined by any of the methods known to the skilled artisan. In some systems, the sequence of each nucleic acid label is determined by sequencing by synthesis or sequencing by hybridization.

In some of the systems described herein, the detection of the nucleic acid label is spatially resolved. Some systems may comprise an electric field generator for the application of an electric field to contact the at least one binding ligand with the intact tissue. Some systems may comprise a micro fluidic chamber.

Systems described herein may be used in the methods described herein.

Provided herein are kits that may be used in the systems and/or methods described herein, said kit comprising: at least one binding ligand that binds a particular protein, wherein said binding ligand is linked to a unique nucleic acid label; a first set of reagents for use when contacting the at least one binding ligand with the tissue sample; and a second set of reagents for use in detection of the nucleic acid label. In some cases the binding ligands are antibodies, or peptide or nucleic acid affinity reagents designed to bind specific proteins as described above.

Some kits may comprise a library of binding ligands, wherein each binding ligand that binds a specific protein is linked to a unique nucleic acid label. Some kits may comprise components for use when the detection is spatially resolved.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings or figures (also “FIG.” and “FIGS.” herein), of which:

FIGS. 1A-1H provide visualization of proteins in a tissue sample comprising cortical white matter tracks from the mouse primary somatosensory cortex and striatal areas, upon contact with DNA-conjugated antibodies and detection labels. FIGS. 1A-1D display that the axon tracks of the white matter are densely stained for tubulin, but have few synapses. FIGS. 1E-IF show that striatal areas are highly enriched in synapses with an increased density of synapsin. FIG. 1A shows alpha tubulin staining in a single section using fluorescently-labeled antisense oligomers against sense oligomers attached to rabbit anti-alpha tubulin primary antibodies via a streptavidin bridge. FIG. 1B shows results of cleavage of the DNA duplex via a restriction site designed into the oligomers. FIG. 1C shows fluorescent anti-rabbit secondary antibodies revealing the location of the primary rabbit antibodies after restriction digestion removed the fluorescent DNA in FIG. 1B. FIG. 1D shows restaining of the same tissue section using a directly-conjugated fluorophore version of the rabbit primary antibody against alpha-tubulin. FIG. 1E shows synapsin staining on the same section as revealed by fluorescently-labeled DNA oligomers complementary to oligomers directly conjugated to rabbit anti-synapsin. FIG. 1E shows that fluorescence seen in FIG. 1F is removed after digestion with restriction endonuclease. FIG. 1G shows fluorescently-labeled secondary antibodies revealing that the primary antibody remains unperturbed after DNA removal. FIG. 1H shows regaining of the same section using anti-synapsin visualized by a secondary anti-rabbit conjugated to a quantum dot.

FIG. 2 provides a composite projection of alpha-tubulin and synapsin in an mated tissue region formed by contacting the tissue and subsequently imaging antibodies that bind alpha-tubulin and synapsin respectively, thereby revealing the axon tracts in the white matter and synapse dense striatal tissue.

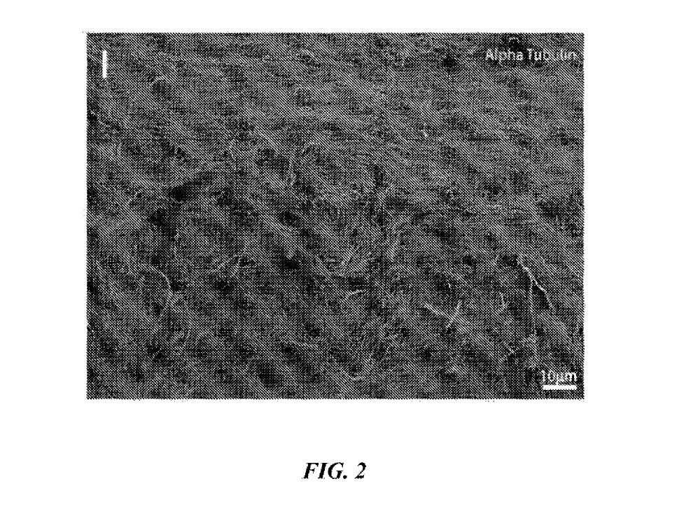

FIG. 3 provides linkers used in DNA sequencing that can be cleaved chemically to liberate the fluorophore.

FIGS. 4A-4C provide images from three different fields of a sample of mouse cortex. The top image. FIG. 4A was acquired in 1.9 s after the sample was incubated with anti-SV2, overnight; the middle image, FIG. 4B, was acquired in 2.2 s after the sample was incubated with anti-SV2 for 10 min in the presence of electric field; the lower image, FIG. 4C, was acquired in 4.4 s after the sample was incubated with anti-SV2 for 10 min, without electric field.

FIG. 5 represents a process.

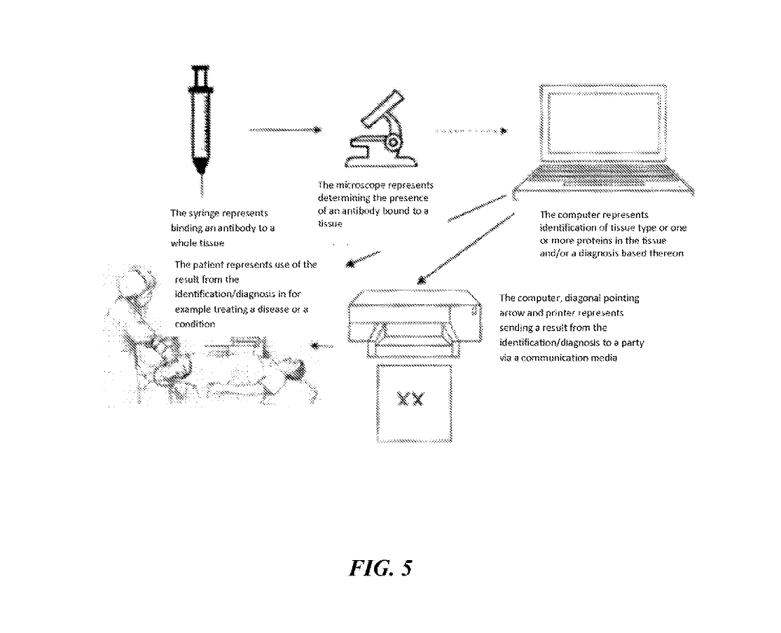

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed. It shall be understood that different aspects of the invention can be appreciated individually, collectively, or in combination with each other.

An object of the present disclosure is to provide methods, systems and kits for performing array tomography on an intact tissue to facilitate spatially resolved identification of a plurality of proteins in the tissue.

Provided herein are methods comprising: contacting an intact tissue sample with at least one binding ligand that binds a particular protein, wherein said binding ligand is linked to a nucleic acid label; and detecting said nucleic acid label, thereby detecting the presence of said protein in the tissue sample. In some cases the binding ligands are antibodies or peptide or nucleic acid affinity reagents designed to bind specific proteins as described above.

Provided herein is a system for identifying the protein composition of an intact tissue comprising: an intact tissue sample; at least one binding ligand that binds a particular protein, wherein said binding ligand is linked to a unique nucleic acid label; and a detector for detection of said nucleic acid label.

Provided herein are kits that may be used in the systems and or methods described herein, said kit comprising: at least one binding ligand that binds a particular protein, wherein said binding ligand is linked to a unique nucleic acid label; a first set of reagents for use when contacting the at least one binding ligand with the tissue sample; and a second set of reagents for use in detection of the nucleic acid label. In some cases the binding ligands are antibodies, or peptide or nucleic acid affinity reagents designed to bind specific proteins as described above.

It maybe noted that the one version of the AT process as currently practiced comprises tissue processing similar to that used for electron microscopy, including chemical fixation, dehydration, and embedding in resin. Tissue blocks are cut on an ultramicrotome using a diamond knife. Contact cement, applied to the block sides, ensures that serial sections stick together to form long ribbons. These are collected on coated coverslips, the coating having been engineered to tightly adhere to embedded tissue sections, holding them fiat for reliable autofocus and retaining them through multiple staining cycles. Arrays are stained using binding ligands, lectins, or other reagents and detected by automated fluorescence microscopy, often at the diffraction limit. Binding ligands, for instance antibodies, can be stripped, and staining and imaging repeated multiple times to build up a high-dimensional data set from a given tissue volume. Arrays can also be stained with heavy metals and imaged by field-emission scanning electron microscopy (SEM). Images are stitched, aligned, and each light (and SEW cycle merged into a 3 D volume comprising all channels. Volumes can be analyzed, for example, to assess the spatial relationships among various markers, providing identification and characterization of synapses, cell types, and other features of interest.

Two aspects of the current process are limiting: 1) to image 4 antigens in a single run, the primary antibodies need to be produced by 4 distinct isotypes, which generally means distinct species, e.g., rabbit, mouse, guinea pig and chicken; 2) the processing required to strip and re-stain the tissue sections is labor-intensive, time-consuming and subject to catastrophic failure.

The first problem could be overcome by directly labeling the primary antibodies with fluorophores. This approach is not common, and only a few directly labeled antibodies are commercially available. A closed chamber microfluidic system could provide a solution to the second problem, and is in fact, described in the methods and systems described herein. The current protocols are, however, complicated, the staining protocol being constituted of approximately 10 steps, including art overnight incubation, and the elution protocol including baking the coverslips, making an automated staining chamber of limited value.

In addition, apart from possible physical damage caused during manual manipulation of the sample, the stripping solution causes chemical damage to the tissue. This is evident in the SEM ultrastructure. The methods and systems described herein eliminate the necessity of stripping the primary antibodies. Further, it provides validated reagents that can be used in combinations of tens-to-hundreds, in a process that can be fully automated.

Unless defined otherwise, all technical and scientific terms used herein have the same meaning as is commonly understood by one of skill in the art to which the claimed subject matter belongs. In the event that there are a plurality of definitions for team herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise.

In the present description, any concentration range, percentage range, ratio range, or integer range is to be understood to include the value of any integer within the recited range and, when appropriate, fractions thereof (such as one tenth and one hundredth of an integer), unless otherwise indicated. As used herein, “about” means+10% of the indicated range, value, sequence, or structure, unless otherwise indicated. It should be understood that the terms “a” and “an” as used herein refer to “one or more” of the enumerated components unless otherwise indicated or dictated by its context. The use of the alternative (e.g., “or”) should be understood to mean either one, both, or any combination thereof of the alternatives. As used herein, the terms “include” and “comprise” are used synonymously.

The section headings used herein are for organizational purposes only and are not to be construed as limiting the subject matter described. All documents, or portions, of documents, cited in the application including, but not limited to, patents, patent applications, articles, books, manuals, and treatises are hereby expressly incorporated by reference in their entirety for any purpose.

It is to be understood that the methods and compositions described herein are not limited to the particular methodology, protocols, methods, and reagents described herein and as such may vary. It is also to be understood that the terminology used herein is for the purpose of describing particular embodiments only, and is not intended to limit the scope of the methods and compositions described herein, which will be limited only by the appended claims.

All publications and patents mentioned herein are incorporated herein by reference in their entirety for the purpose of describing and disclosing, for example, the constructs and methodologies that are described in the publications, which might be used in connection with the methods, compositions and compounds described herein. The publications discussed herein are provided solely for their disclosure prior to the filing date of the present application. Nothing herein is to be construed as an admission that the inventors described herein are not entitled to antedate such disclosure by virtue of prior invention or for any other reason.

Intact Tissue:

The methods and systems described herein may be used to perform array tomography on an intact tissue sample. An intact tissue as described herein includes tissues that are sectioned on one dimension and contiguous in the other two dimensions. These tissues are characterized by minimal dissociation. An intact tissue sample is one wherein after sectioning, the sample retains tissue architecture and other cells normally found in the whole tissue. Exemplary methods of fixing intact tissue for the methods and systems described herein are provided in Example 1 below. Additionally methods of isolating and fixing intact tissue samples known to the skilled artisan can be employed for the methods and systems described herein. In some of the methods described herein, the intact tissue may be embedded in a resin such that the tissue can be sliced into sections of thickness between 20 and 1000 nm. In some methods, the thickness of the section may be 25, 30, 35, 40, 45, 50, 55, 60, 70 80, 90 100, 150, 200, 250, 300, 350, 400, 450, 500, 550, 600, 650, 700, 750, 800, 850, 900, 950 or 1000 nm. In some methods, the thickness of the section may be about 100, 150, 200, 250, 300, 350, 400, 450, 500, 600, 700, 800, 900 or 1000 nm. In some cases, the method may not comprise dehydration of the intact tissue. In some cases, the tissue is not dehydrated and resin embedded. In some cases, a section collector is utilized to automatically collect ribbons produced on an ultramicrotome and place them on pre-defined regions of coated, precision coverslips, of sizes ranging from a microscope slide to a microtiter plate. Intact tissue samples that can be studied by this method may include for instance biopsied tissues for detection of one or more conditions. Physiological conditions or diseases maybe diagnosed by the methods provided herein by the identification of proteins associated with such conditions or diseases in the intact tissue sample. These include for instance detection of kidney diseases such as crescentic glomerulonephritis, infectious diseases that maybe diagnosed by studying biopsied lymph node tissue, metabolic diseases including amyloidosis, and fertility levels as may be detected from testicular biopsies. Pre-cancerous and cancerous conditions can be identified by applying the methods described herein to biopsied intact tumor tissues. Other tissues that are generally studied by biopsies can be analyzed by the methods and systems described herein, for instance, bone marrow, gastrointestinal tract, lung, liver, prostate, nervous system, urogenital system, breast, muscle and skin.

### Array Tomography of Intact Tissue:

Provided herein are methods comprising: contacting an intact tissue sample with at least one binding ligand that binds a particular protein, wherein said ligand is linked to a nucleic acid label; and detecting said nucleic acid label, thereby detecting the presence of said protein in the tissue sample. In an aspect, the methods may comprise contacting the intact tissue with a plurality of binding ligands, wherein each ligand that binds a specific protein is linked to a unique nucleic acid label. In some cases each ligand in the plurality of binding ligands may bind a different protein. In some cases each binding ligand is an antibody, or peptide or nucleic acid affinity reagents designed to bind a specific protein as described above. The ligands may be of the same or different isotypes, and the nucleic acid labels may comprise DNA and/or RNA. In some cases ligands that bind different proteins may be of the same or different isotypes. In some methods, the at least one binding ligand maybe cross-linked to the tissue. In some cases, a method described herein may comprise contacting the intact tissue with a plurality of binding ligands, wherein each binding ligand and that binds a specific protein is linked to a unique nucleic acid label. In some cases the binding ligands are antibodies, or peptide or nucleic acid affinity reagents designed to bind specific proteins as described above.

In methods described herein, the detection of the plurality of binding ligands may be spatially resolved. Some of the methods and systems described herein comprise use of a microfluidic chamber. Some methods may be fully automated.

Some methods described herein may be used to identify the protein composition of the tissue sample, and/or diagnose a physiological condition or disease as described above. Some methods maybe used to identify the tissue class of a particular intact tissue.

In some of the methods described herein, contacting the tissue with a binding lip and may comprise application of an electric field. In some cases, the electric field may be applied for about 1, 2, 3, 4, 5, 6, 7, 8, 9, 10, 12, 15, 20, 25, 30, 35, 40, 45, 50, 55, or 60 seconds. In some cases, the electric field maybe applied for between 0.5, 1, 1.5, 2, 2.5, 3, 3.5, 4, 4.5 or 5 minutes. In some cases, the electric field maybe applied for up to 5, 10, 15, 20, 25, 30, 35, 40, 45, 50, 55, 60, 65, 70, 75, 80, 85, or 90 minutes. In some cases, the electric field maybe applied between 1 and 60 minutes.

Also provided are systems suitable for carrying out the methods described herein, and kits for use with such systems.

Detection Labels and Detection Tags:

In some of the methods described herein, detection comprises exposing the tissue to detection labels unique for each nucleic acid label. Each detection label may comprise at least one nucleic acid oligomer comprising a sequence which is complementary to a sequence of at least one nucleic acid label. In some cases, the detection label comprises at least one detection tag. In some eases, the detection label may also comprise a plurality of detection tags. In some eases, the detection label may comprise between 1, 2, 3, 4, 5, 6, 7, 8, 9, 10, 11, 12, 13, 14, 15, 16, 17, 18, 19 and 20 detection tags.

In some cases one or more of the detection tags may be attached to the oligomer. A detection tag may be attached to an oligomer by a cleavable or a non-cleavable linker. In some cases a plurality of detection tags are attached to the oligomer, each spaced between seven and thirty bases apart from each other. In some cases, the plurality of detection tags are attached such that each tag is spaced between 2, 3, 4, 5, 6, 7, 8, 9, 10, 11, 12, 13, 14, 15, 16, 17, 18, 19, 20, 21, 22, 23, 24, 25, 26, 27, 28, 29 and 30 bases apart. In some instances, the plurality of detection tags may comprise between two and ten detection tags. In some cases, the plurality of detection tags may comprise between 1, 2, 3, 4, 5, 6, 7, 8, 9, 10. 11, 12, 13, 14, 15, 16, 17, 18, 19 and 20 detection tags.

In some of the methods described herein, each detection tag may comprise afluorescent tag. In some cases the fluorescent tag may be a quantum dot (QD) as described in the examples below. In some cases the fu scent tag may be at least one fluorescent dye. In some exemplary instances, the fluorescent dye may comprise at least one of coumarin, rhodamine, xanthene, fluorescein and cyanine. In general any fluorescent dye and/or QD known to the skilled artisan may be employed in the methods and systems described herein. In some methods and systems described herein, the placement and number of detection tags may be optimized to enhance spatial resolution of the detection.

In some of the methods described herein, hybridization of the detection tag with the nucleic acid label may comprise application of an electric field. In some cases, the electric field may be applied for about 1, 2, 3, 4, 5, 6, 7, 8, 9, 10, 12, 15, 20, 25, 30, 35, 40, 45, 50, 55, or 60 seconds. In some cases, the electric field maybe applied for between 0.5, 1, 1.5, 2, 2.5, 3, 3.5, 4, 4.5 or 5 minutes. In some cases, the electric field maybe applied for up to 5, 10, 15, 20, 25, 30, 35, 40, 45, 50, 55, 60, 65, 70, 75, 80, 85 or 90 minutes. In some cases, the electric field maybe applied between 1 and 60 minutes.

Some of the methods described herein may not comprise the detection of a secondary antibody.

Also provided are systems suitable for carrying out the methods described herein, and kits for use with such systems.

Detection by Sequencing:

In some cases, the methods described herein, may comprise a detection step that comprises determining the sequence of each nucleic acid label. In general any sequencing method that can be performed in-situ can be utilized for sequencing the nucleic acid labels herein. These include for instance sequencing by synthesis, sequencing by ligation, sequencing by hybridization among other methods known to the skilled artisan. Commercially available nucleic acid sequencing kits may be optimized for use with the methods and systems described herein.

In some cases the sequence of each nucleic acid label may be determined by sequencing by synthesis. In some instances, the sequence of each nucleic acid label may be determined by sequencing by hybridization. Sequencing by hybridization may involve use of the tag hybridization method described in the examples below.

Tag sequencing is a variant of direct sequencing uses tags that are about 60 base pairs (bp) consisting of 4 about 15 mer units as described in the examples below, in some cases each oligomer is 9, 10, 11, 12, 13, 14, 15, 16, 17, 18, 19, or 20 nucleic acids in length. In some cases, tag ‘sequencing’ by hybridization is used with. QD-labeled oligomers. Using QDs enables reasonably high-speed STORM-like imaging. Further, the quantum dots do not need to be photo-activated, are resistant to photobleaching, and require a single color for excitation. In some cases, tag sequencing is used with cleavable fluorescent labels.

Also provided are systems suitable for carrying out the methods described herein, and kits for use with such systems.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims.

The reagents employed in the examples are commercially available or can be prepared using commercially available instrumentation, methods, or reagents known in the art. The foregoing examples illustrate various aspects of the invention and practice of the methods of the invention. The examples are not intended to provide an exhaustive description of the many different embodiments of the invention. Thus, although the forgoing invention has been described in some detail by way of illustration and example for purposes of clarity of understanding, those of ordinary skill in the art will realize readily that many changes and modifications can be made thereto without departing from the spirit or scope of the appended claims.

### Example 1

### Tissue Preparation

Generally tissue preparation methods outlined in Micheva, K. D., O'Rourke, N., Busse, B., & Smith, S. J. (2010). Array Tomography: Rodent Brain Fixation and Embedding. Cold Spring Harbor Protocols. 2010 (11), are used (by adapting and optimizing for the difference in the tissue type and the organism from which the tissue is obtained).

Mouse brain was dissected and fixed as follows:

Dissection tools were set up and PBS and filtered fixative were prepared, ready to flow, without air bubbles. 2% glutaraldehyde and 2% depolymerized paraformaldehyde, were dissolved in 0.1 M phosphate buffer, with pH between 6.8 and 7.2.

The rodent was anesthetized without killing, the heart was exposed and the right atrium was cut and a cannula inserted into the left ventricle. A blunted ˜20 G needle, shortened to about 1 cm was used. In some cases, for organisms where the aorta is not fragile or easily destroyed, it is optimal to cumulate the aorta. The fixative was then allowed to flow for about 10 minutes by use of gravity flow; or in some instances a perfusion pump. This was then perfused with up to 5 ml of PBS.

About 5 cc heparinized saline was then put in the tube, to help flush blood. Perfusion with fixative was performed for 10 minutes. The brain was removed within 20 minutes of fixation. The whole brain was postfixed in the same fixative overnight in the. refrigerator. After rinsing 2× in 0.1 M Phosphate buffet, the tissue was stored up to one week at 4° C.

In certain cases, thick tissue sections are analyzed, approximately 200 nm-1 □m for resin-embedded tissue. It is noted that thick sections allow imaging larger volumes per unit time. Imaging may be performed at high magnification or with relatively low magnification objectives, 10-20×. For instance in the analysis of biopsied tissues such as tumors, it may be preferred to perform analysis of thick sections at lower magnifications. The lower magnification allows analysis of large fields of tissue with subcellular resolution.

In some cases, the tissue is not dehydrated and resin embedded, rather the labeling methods described below are applied to binding ligands that have been validated for staining of fixed, hydrated tissue.

In some cases, a section collector is utilized to automatically collect ribbons produced on an ultramicrotome and place them on pre-defined regions of coated, precision coverslips, of sizes ranging from a microscope slide to a microliter plate.

### Example 2

### Protein Detection by Contacting Intact Tissue Sample with an Antibody Linked to a Nucleic Acid Label That Binds to a Detection Label Comprising a Nucleic Acid Oligomer

Described below is the use of the methods described herein for the detection of proteins tubulin and synapsin in an array tomography (AT) intact tissue sample from a mouse by contacting with an antibody that is linked to a nucleic acid label that binds to a detection label comprising a nucleic acid oligomer that is complementary to the nucleic acid label. The oligomer is attached to one or more fluorophores or quantum dots (QDs) to facilitate detection.

A tissue sample was prepared by the method described in Example 1.

Antibody Labeling with Nucleic Acid Label Attached by Streptavidin Bridge:

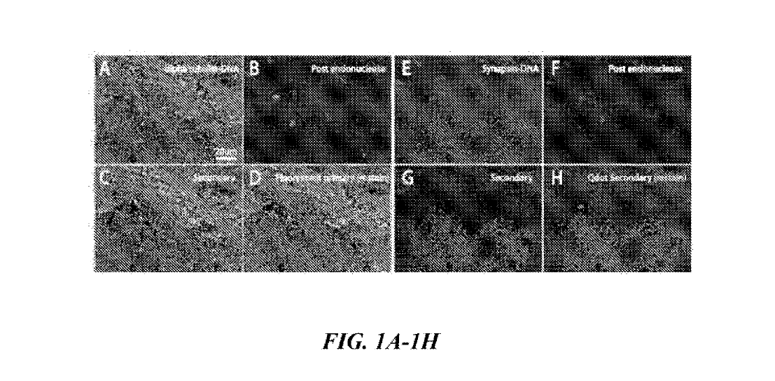

A rabbit anti-alpha tubulin antibody (primary antibody) was introduced to the tissue sample, which was then contacted with streptavidin, followed by a biotinylated nucleic acid label, and then a fluorescently labeled antisense oligomer which was complementary to the sense oligomer attached to the primary antibody. As seen in FIG. 1A, this resulted in good staining of the tubulin in the tissue sample.

In one example, the nucleic acid label on the primary antibody was designed with a recognition site for a restriction enzyme (Smal). In this case, after the detection label comprising the complementary nucleic acid oligomer hybridized with the nucleic acid label and was imaged, the dsDNA funned by the hybridization of the complementary oligomers was cleaved by the restriction enzyme, releasing the fluorescent label along with the short piece of dsDNA. As seen in FIG. 1B, this demonstrated that the AT tissue was permissive for the DNA hybridization reaction and that the reaction was specific.

The tissue was then exposed to a fluorescently labeled secondary antibody that bound to the primary anti-tubulin antibody. FIG. 1C shows the fluorescent anti-rabbit secondary antibody revealing the location of the primary rabbit tubulin antibodies.

Antibody Labeling with Nucleic Acid Label Attached Without Use of Streptavidin Bridge:

In another example, the tissues section used for the above experiments was exposed to rabbit anti-synapsin antibody which was directly conjugated to a nucleic acid label comprising a sense oligomer, and subsequently exposed to fluorescently-labeled complementary DNA antisense oligomer. FIG. 1E shows synapsin staining on the same section as revealed by fluorescently-labeled DNA oligomer complementary to an oligomer directly conjugated to rabbit anti-synapsin. As seen in FIG. 1F, the fluorescence was removed when the resulting dsDNA was digested by use of a restriction endonuclease. Subsequent introduction of a fluorescently-labeled secondary antibody resulted in its binding to the anti-synapsin primary antibody. FIG. 1G shows the fluorescent anti-rabbit secondary antibody revealing the location of the primary rabbit synapsin antibodies. FIG. 1H demonstrates staining of the tissue section using anti-synapsin visualized by a secondary anti-rabbit conjugated to a quantum dot (QM It is noted that the emission spectra of QDs are narrower than typical dye fluorescence, making it possible to increase the number of detection channels per run from 4 to 6, or more. This reduces the overall imaging time for large data sets.

The nucleic acid labels were separately conjugated to the anti-synapsin antibodies using the Solulink All-in-one-antibody-oligonucleotide-conjugation kit and the Inflow Biosciences Thunder-link oligo conjugation system.

Imaging was perforated at 63×, 1.4 NA. Exposure times were approximately 1 s. Detection oligos were purchased from IDT.

In some tissue structures such as pre-synaptic terminals, the crowded structure limits the number of binding ligands that can bind. In these cases, it is tolerable to have reduced signal associated with the detection of each protein in exchange for a corresponding increase in the number of proteins that can be identified, with improved quantitation of their relative amounts.

In some cases, detection labels comprising complementary oligomers labeled with multiple fluorophores are used. To obtain increased brightness from the successive fluorophores, the fluorophores are on average spaced 8-10 bases apart. In some eases the optimized detection oligomers have about 3 to 6 fluorophores.

It is noted that in some cases the detection label comprises one or more quantum dot (QD) linked to the complementary nucleic acid sequence. QDs used in these labels are inorganic nanocrystal semiconductors that behave exceptionally well as fluorophores. In some cases cadmium-free QDs are utilized, while in some other cases the QDs have a CdSe core. QDs with a CdSe core may have a ZnS shell and/or may be encapsulated in a hydrogel. The emission spectra of QDs are relatively narrower than typical fluorescence dyes, allowing detection of about six distinct QD-labeled binding ligands in a single imaging run, on most tissue types.

### Example 3

### Protein Detection by Contacting Intact Tissue Sample with an Antibody Linked to a Nucleic Acid Label Followed by Sequencing of the Nucleic Acid Label

Described below is the use of the methods described herein for the detection of protein synapsin in an array tomography (AT) intact tissue sample from a mouse by contacting with an antibody that is linked to a nucleic acid label that is then detected by sequencing.

A tissue sample is prepared by the method described in Example 1. The tissue sample is then exposed to rabbit anti-synapsin antibody which is directly conjugated to a nucleic acid

The nucleic acid label is then identified by sequencing.

While detecting a plurality of proteins, the use of sequencing has some inherent advantages. For instance, detection of 100 proteins in a tissue sample, read 4 at a time using the method described in Example 2, requires 25 imaging runs; whereas direct sequencing of the unique nucleic acid labels of the binding ligands would require only ˜4 reads. Because the order of the bases is known, 4 bases present 44=256 combinations, and even if the restriction is applied that the same base cannot occur in succession, the number of possible sequences for a 4 mer is 4×3×3×3=108.

DNA Sequencing by Synthesis:

In general DNA sequencing by synthesis is performed by the methods and protocols outlined in Bentley, D. R., et. al. (2008). Accurate whole human genome sequencing using reversible terminator chemistry. Nature, 456 (7218), 53-59.

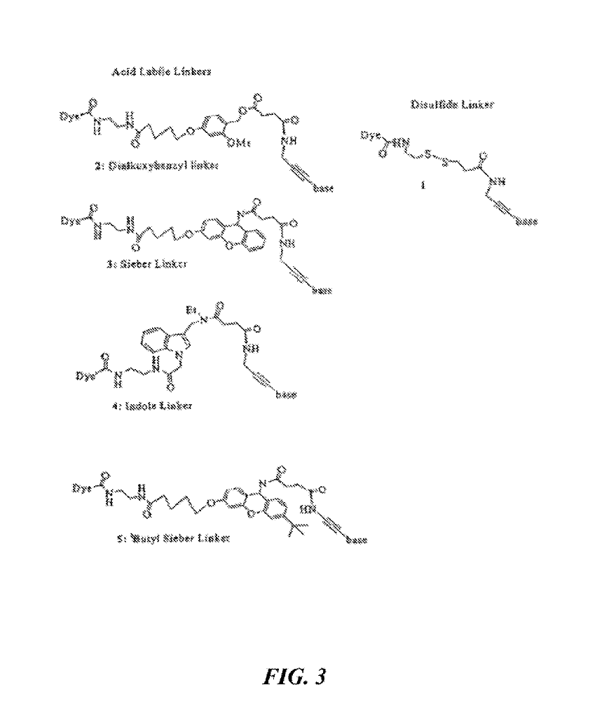

FIG. 3 shows various linkers used in DNA sequencing that are cleaved chemically to liberate a fluorophore. Fluorescent labels are removed by enzymatic or chemical cleavage. There are numerous chemically cleavable linkers that are used to attached fluorophores to nucleotides. Other chemistries including azides and allyl groups, are used as well. Photocleavable linkers have also been demonstrated. Labeled oligomers having disulfide linkers are used as available from Trilink. Cleavage is accomplished by addition of TCEP-HCl [Tris(2-carboxyethyl)phosphine hydrochloride], a compound that selectively and completely reduces even the most stable water-soluble alkyl disulfides over a wide pH range in about 5 minutes at room temperature.

DNA Sequencing by Hybridization (Tag ‘Sequencing’)

In some cases, tag sequencing by hybridization is employed. This is a variant of direct sequencing uses tags that are about 60 base pairs (bp) consisting of 4 about 15 mer units. With this approach, the number of unique combinations is 4^{n.} or 256 for n=4. For the detection of for instance 100 proteins in a tissue sample, each of the 100 binding ligands has a tag consisting of 4 unique 15 mers (corresponding to A, T, C or G) at each of the positions, requiring 16 unique oligomers, in total. The ‘sequencing’ could be from either end. All that is required is that when sequencing position m, the 4 oligomers that are complementary to the tag oligomers in position m are introduced. For example, oligomers, each labeled with a distinguishable fluorophore, complementary to the 4 unique sequences on the distal end of the tags, are introduced and read out; the fluorophores are then removed, either by cleaving the linker, or by enzymatically cleaving the dsDNA to release the fluorophore. The latter method requires sequencing from the distal end. Because this tag-sequencing scheme allows using each fluor in each round, the formula for the number of unique tags is pⁿ (above we assumed p=4). Using QDs allows increasing p, from 4 to 6, or more.

Tag ‘sequencing’ by hybridization works well with QD-labeled oligomers. Assuming p=6, and n=3, 216 binding ligands could be uniquely labeled, using only 6×3=18, unique oligomers and 3 reads. Using QDs enables reasonably high-speed STORM-like imaging. It has been demonstrated that one can take advantage of quantum dot blinking to obtain three-dimensional super-resolution imaging with ˜15 rim in the plane. Further, the quantum dots do not need to be photo-activated, are resistant to photobleaching, and require a single color for excitation.

### Example 4

### Use of a Microfluidic Chamber

In some cases, the detection methods described above are implemented in an automated instrument comprising a microfluidic chamber. A ‘section collector’ automatically collects ribbons produced on an ultramicrotome and places them on pre-defined regions of coated, precision coverslips, of sizes ranging from a microscope slide to a microtiter plate. Chambers are formed by adding a ‘top-piece’ designed with ports that are to be accessed either by a pipetting robot, or coupled to fittings so as to from a closed microfluidic system.

In some cases is a microfluidic chamber that processes a method using a separate detection oligomer for each binding ligand, which requires a valve system that multiplexes 100 reagents for instance while detecting 100 proteins, as well as the cleavage and wash solutions. This is accomplished by connecting the Chamber to a 10-input, 1-output valve and connecting each input to a similar valve. The 2-level system could handle 100 separate solutions.

In some cases, the microfluidic chamber is constructed with transparent, or semi-transparent electrodes on the top and bottom of the chamber in order to facilitate the application of a suitable electric field. Suitable materials for the electrodes include indium-tin-oxide (ITO), carbon and gold.

### Example 5

### Application of an Electric Field

In some cases, an electric field is applied to reduce the amount of time needed for contacting each binding ligand to the tissue sample.

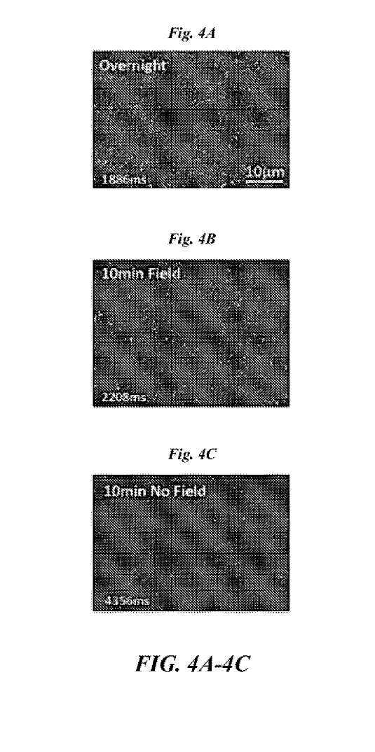

For instance FIGS. 4A-4C display images from three different fields of a sample of mouse cortex. FIG. 4A was acquired in 1.9 s after the sample was incubated with anti-SV2, overnight; FIG. 4B was acquired in 2.2 s after the sample was incubated with anti-SV2 for 10 min in the presence of electric field; and FIG. 4C was acquired in 4.4 s after the sample was incubated with anti-SV2 for 10 min, without electric field. The sample was placed on a carbon-coated coverslip. A chamber was formed by the sample coverslip and another carbon-coated coverslip positioned parallel at an offset of ˜0.5 mm. The applied voltage between the opposing coverslips was 150V.

In cases comprising hybridization of oligomers as in some Examples described above, the hybridization time is reduced from 15-30 min to approximately 1 minute by the application of an electric field. In some cases a short reverse pulse is additionally applied to increase specificity by removing unbound oligomers.

### Example 6

### Localization of RNA Binding Ligands

Nucleic acid-tagged ligands as described above are used for protein extraction for subsequent analysis with mass spectrometry and/or RNA sequencing. For instance, a binding ligand that binds to an RNA binding protein and a ligand that localizes or binds to a particular cellular compartment, or to a particular protein complex, are applied to an intact tissue sample. The ligands are labeled with complementary oligomers that can form a DNA bridge and with linkers further comprising a QD and a magnetic bead. Imaging the QD emission provides confirmation that the ligand complex is localized to the appropriate compartment in the cell. Following formation of the bridge and treatment with a single-strand DNAse, the ligands are fixed to the tissue, the tissue is disrupted and the protein complex extracted with the magnetic beads. The protein complex is analyzed with mass spectrometry or RNAseq.

In some cases, two ligands (A and B) each labeled with double-stranded DNA, one with a T overhang the other with an A overhang are introduced to the tissue sample. A ligase is applied and the TA base pairing allows ligation if the two ligands are sufficiently close, analogous to TA cloning. One ligand is conjugated to a magnetic bead; the other is conjugated to biotin. The tissue is disrupted and the labeled protein complexes extracted in magnetic and streptavidin purification procedures. This produces 3 groups of tissue (A only, B only and A B) for further analysis.

It is to be understood that the terminology used herein is used for the purpose of describing specific embodiments, and is not intended to limit the scope of the present invention. It should be noted that as used herein, the singular forms of “a”, “an” and “the” include plural references unless the context clearly dictates otherwise. In addition, unless defined otherwise, all technical and scientific terms used herein have the same meaning as commonly understood by one of ordinary skill in the art to whish this invention belongs.

While preferable embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention describe herein may be employed in practicing the invention. It is intended that the following claims define the scope of the invention and that methods and structures within the scope of these claims and their equivalents be covered thereby.

What is claimed is:

1. A method comprising:
contacting an intact tissue sample with at least one binding ligand that binds a particular protein, wherein said at least one binding ligand is linked to a nucleic acid label; and
detecting said nucleic acid label, thereby detecting the presence of said particular protein in said intact tissue sample.

2. The method of claim 1, comprising contacting said intact tissue sample with a plurality of binding ligands, wherein each binding ligand that binds a particular protein is linked to a unique nucleic acid label.

3-5. (canceled)

6. The method of claim 1, wherein the method comprises embedding said intact tissue sample in a resin such that said intact tissue sample is sliced into sections of thickness between 20 and 1000 nm.

7. The method of claim 1, wherein said nucleic acid label comprises a nucleic acid oligomer.

8. (canceled)

9. The method of claim 1, further comprising contacting said intact tissue sample to a detection label that comprises a nucleic acid oligomer, wherein said detection label comprises a sequence which is complementary to a sequence of said nucleic acid label.

10. The method of claim 9, wherein said detection label comprises at least one detection tag.

11-12. (canceled)

13. The method of claim 1, wherein said binding ligand is linked to said nucleic acid label by a non-cleavable linker.

14-20. (canceled)

21. The method of claim 1, wherein detecting comprises determining a sequence of each said nucleic acid label by sequencing by synthesis or sequencing by hybridization.

22-27. (canceled)

28. The method of claim 1, wherein contacting said intact tissue sample with said ligand comprises application of an electric field.

29-31. (canceled)

32. A system for identifying the protein composition of an intact tissue comprising:
an intact tissue sample;
at least one binding ligand that binds a particular protein in said intact tissue sample, wherein said binding ligand is linked to a unique nucleic acid label; and
a detector for detection of said nucleic acid label.

33. The system of claim 32, wherein said intact tissue sample is resin embedded and has a section of thickness between 20 and 1000 nm.

34. The system of claim 32, wherein said nucleic acid label comprises a nucleic acid oligomer.

35. The system of claim 32, comprising a plurality of binding ligands, wherein each said binding ligand that binds a specific protein is linked to a unique nucleic acid label.

36. (canceled)

37. The system of claim 32, further comprising a detection label that comprises a nucleic acid oligomer, wherein said detection label comprises a sequence which is complementary to a sequence of said nucleic acid label.

38. The system of claim 37, wherein said detection label comprises at least one detection tag.

39. The system of claim 32, wherein said binding ligand is linked to said nucleic acid label by a non-cleavable linker.

40. The system of claim 32, wherein said detection comprises determining the a sequence of each nucleic acid label by sequencing by synthesis or sequencing by hybridization.

41-42. (canceled)

43. The system of any of claim 32, further comprising an electric field generator for the application of an electric field during contact of said at least one binding ligand with said intact tissue sample.

44-45. (canceled)

46. The system of claim 32, wherein said at least one binding ligand is selected from ankyrin repeat proteins, anticalins, cysteine knottin scaffolds, cyclic peptides, fynomers, affitins, sso7d, fibronectins, affibodies, and Gp2 protein or fragments thereof.

47. A kit, said kit comprising:
at least one binding ligand that binds a particular protein in an intact tissue sample, wherein said binding ligand is linked to a unique nucleic acid label;
a first set of reagents for use when contacting the at least one binding ligand with said tissue sample; and
a second set of reagents for use in detection of said nucleic acid label.

48-49. (canceled)
